(19) United States
(12) Patent Application Publication
(10) Pub. No.: US 2022/0008598 A1
(43) Pub. Date: Jan. 13, 2022

(54) Antibacterial & Antiviral Decontamination System

(71) Applicant: Gary Kohring, Phoenix, AZ (US)

(72) Inventor: Gary Kohring, Phoenix, AZ (US)

(21) Appl. No.: 17/124,828

(22) Filed: Dec. 17, 2020

Related U.S. Application Data
(60) Provisional application No. 63/048,735, filed on Jul. 7, 2020.

(51) Int. Cl. A61L 9/14; A61L 9/013

(52) U.S. Cl. CPC A61L 9/14; A61L 9/013; A61L 2101/32; A61L 2209/135; A61L 2209/21; A61L 2209/16

(57) ABSTRACT

The present disclosure relates to a unique and novel antibacterial and antiviral decontamination system that can be integrated into an existing HVAC system in a vehicle, and that can be used to decontaminate and sanitize the interior of the vehicle in a safe, efficient and thorough manner. The system may be controlled manually via a switch within the cabin of the vehicle, or remotely via, for example, a smart device. The system comprises a replaceable and refillable cartridge that contains an antibacterial, antiviral, sanitizing solution, and a vaporizer that is used to vaporize the solution into a fine mist that is then dispersed throughout the interior of the vehicle via the existing HVAC system.

### CROSS-REFERENCE TO RELATED APPLICATION

The present application claims priority to, and the benefit of, U.S. Provisional Application No. 63/048,735, which was filed on Jul. 7, 2020 and is incorporated herein by reference in its entirety.

### FIELD OF THE INVENTION

The present invention relates generally to the field of sterilization of the interior area of commercial and personal vehicles. More specifically, the present invention relates to a disinfecting system to decontaminate such areas and surfaces relatively quickly to reduce and eliminate the transmission of viruses, bacteria and other pathogens. The disinfecting system of the present invention releases an antibacterial and antiviral decontamination mist to decontaminate the interior of a vehicle, and is integrated directly into the HVAC and ventilation system of the vehicle. The disinfecting system includes a cartridge filled with an antiviral, antibacterial and sanitizing solution which, upon release from the HVAC and ventilation system, impacts the entire interior area of the vehicle. A switch is present at a convenient location for the user, such as the dashboard of the vehicle, to activate the disinfecting system. Once activated, the disinfecting system dispenses the solution as a fine mist throughout the interior of the vehicle to sanitize the same. The disinfecting system can be activated once the existing passengers exit the vehicle, and prior to new passengers coming on board, such as after an airplane flight, aboard a rental car, or in another situation where different individuals may utilize the same interior compartment. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, pathogens, viruses, bacteria, and the like are easily transmitted from person to person in confined spaces. This is particularly true because confined spaces do not allow individuals therein to maintain a minimum safe distance among themselves due to the limited space. Accordingly, people having an infectious disease, bacteria, virus, or other harmful pathogens could easily contaminate an entire passenger cabin of a train, airplane, bus or other vehicle. More specifically, individuals travelling in an airplane, train, bus or the like are more vulnerable to being exposed to such unwanted pathogens, as such individuals are confined closely together, breathing the same air and touching the same surfaces.

While mass transportation vehicles, such as airplanes, trains, buses, etc. are periodically sanitized, doing so requires a lot of manual labor and effort. Specifically, the process of sterilizing the complete seating compartment consumes a lot of time and may be ineffective in eliminating all of the germs, bacteria, viruses, and the like, particularly from hard to reach places, and may result in some spots being left unclean. This, in turn, can lead to the spread of germs, bacteria, and dangerous viruses. Further, manual cleaning and sanitizing efforts suffer from a number of additional challenges including, without limitation, re-contamination of surfaces from dirty sponges and mops, the improper use and mixing of anti-bacterial cleaning solutions, etc. Therefore, adequate and thorough decontamination of the seating compartment of a commercial vehicle is nearly impossible.

Further, it is also difficult to sterilize the interior area of, for example, airplanes between different flights, or trains between different trips, as usually there is very less time in between the two events to conduct a proper cleaning. Less time to clean the vehicle contributes to the ineffective sterilization of the interior space of the vehicle, and increases the likelihood of the transmission of a virus, bacteria or other harmful pathogens.

Therefore, there exists a long felt need in the art for an antibacterial and antiviral decontamination misting device that can be employed within vehicles, such as airplanes, trains, buses, cabins, taxis, personal vehicles and more, and that creates a spray designed to cover the entirety of the confined space and effectively eliminate bacteria, germs, and viruses therefrom. There is also a long felt need in the art for an antibacterial and antiviral decontamination misting device that evenly spreads the decontamination spray over all surfaces, and cleans the complete vehicle thoroughly. Moreover, there is a long felt need in the art for an antibacterial and antiviral decontamination misting device that can be easily integrated into the existing HVAC and/or ventilation system of the vehicle, and that does not require a new infrastructure for installation or use. Additionally, there is also a long felt need in the art for an antibacterial and antiviral decontamination misting device that provides immediate disinfection (e.g., between flights or train departures), and that eliminates most of the manual labor and time otherwise required to disinfect the same, thereby enabling travelers to maintain proper sanitary conditions. Finally, there is a long felt need in the art for an antibacterial and antiviral decontamination misting device that is relatively inexpensive to manufacture and is both safe and easy to use.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a unique disinfecting system integrated into the HVAC and/or ventilation system of a vehicle. The disinfecting system preferably comprises a cartridge filled with an antibacterial and antiviral solution, and a distribution system integrated directly into the HVAC and/or ventilation system of the vehicle for distributing the antibacterial and antiviral solution throughout the vehicle. More specifically, a switch is present on an easily accessible location in the vehicle, such as on a dashboard, to send a trigger signal to the cartridge to release the antibacterial and antiviral solution throughout the distribution system. Once activated, the system dispenses the antibacterial and antiviral solution as a fine mist through the vents of the vehicle to sanitize the interior of the vehicle thoroughly and completely.

In a further embodiment of the present invention, a method of disinfecting an interior of a vehicle is provided. The method includes the steps of initially vacating the interior of the vehicle to make sure that no passenger is present in the vehicle, and then switching on or otherwise activating the disinfecting system by using a switch present on the dashboard of the vehicle. Next, an antibacterial and antiviral decontamination solution is released through a distribution system of the disinfecting system, wherein the distribution system is integrated into the HVAC and/or ventilation system of the vehicle. The antibacterial and antiviral decontamination solution is dispensed through the distribution system as a relatively fine mist to sanitize the entire interior area and surfaces of the vehicle. Finally, the release of the sanitizing mist is automatically discontinued after a pre-determined period of time, or may be manually discontinued through the switch present on the dashboard.

The antibacterial and antiviral solution mist that is released from the HVAC and ventilation system into the vehicle compartment sterilizes the surfaces and destroys the bacteria and other pathogens in the duct work and vehicle interior, and eliminates daily odors and mixed odors (such as cigarettes, pet odors, etc.) as well. The mist is produced by a vaporizer present in the disinfecting system which receives the antibacterial and antiviral solution from the cartridge. The mist is, in turn, released through the HVAC and/or ventilation system of the vehicle. The vaporizing system may be an ultrasonic system or any other suitable mist or vapor emitting system. In one embodiment, a display system is associated with the disinfecting system and displays the status of the operation of the disinfecting system, such as “Disinfection in progress” or “Please do not enter”. An audible message, such as alarm, may also be emitted by the disinfecting system to create audible notifications for the user to notify the user when the disinfection process is taking place.

In a further embodiment of the present invention, a disinfecting system for sanitizing an interior of a vehicle using the vehicle's existing HVAC and ventilation system is disclosed. The disinfecting system comprises a sensor to detect the presence of a human, a cartridge containing an antibacterial and antiviral decontamination solution, and a distribution system integrated within the HVAC and ventilation system of the vehicle to disperse the antibacterial and antiviral decontamination solution throughout the interior of the vehicle. The disinfecting system may further comprise a mist dispenser to dispense the antibacterial and antiviral decontamination solution as a mist in the interior of the vehicle when no human activity is detected in the interior of the vehicle.

In a further embodiment of the present invention, a passenger cabin sanitizing system is disclosed which uses the existing HVAC and ventilation system of the passenger cabin. The cabin sanitizing system comprises a cartridge containing an antibacterial and antiviral decontamination solution, wherein the cartridge has a nozzle to release the antibacterial and antiviral decontamination solution to a distribution system. The distribution system, in turn, carries the antibacterial and antiviral decontamination solution to a vaporizer to create a sanitizing mist that is released through the existing HVAC and vents of the passenger cabin. A switch is used to activate the passenger cabin sanitizing system, which is connected to the vehicle's Low Direct Current Voltage power supply.

In yet a further embodiment of the present invention, a method of sanitizing a passenger cabin of a commercial vehicle, such as airplane, bus, van, train or the like, is disclosed. The method comprises having people exit the passenger cabin, then sealing the passenger cabin such that air inside the passenger cabin is substantially prevented from exiting the passenger cabin. Next, switching on the disinfecting system of the present invention by operating a manual switch to release a mist of an antibacterial and antiviral decontamination solution into the passenger cabin. The antibacterial and antiviral decontamination solution is stored in a cartridge and, when released, sanitizes the interior space and surfaces of the passenger cabin for at least a period of time equal to an appropriate sanitation period, and the opening of the passenger cabin for the passengers.

In one embodiment, the antibacterial and antiviral decontamination solution may include hydrogen peroxide along with various fragrances. The antibacterial and antiviral decontamination solution is preferably a medical-grade contact disinfectant that destroys 99.99% of the germs responsible for cold and flu, bacteria and viruses such as staph, methicillin resistant staphylococcus (MRSA), COVID-19 and legionella. In addition, the solution mist prevents mold and fungus growth. The hydrogen peroxide is provided in solution ranging from about 2% to about 10% by weight of the solution. The remaining amount of solution includes water ranging from about 80% to about 95%. Fragrances such as essential oils may range from 2-8% of the solution by weight. Essential oils may include lavender, eucalyptus, ginger, clove, cedar wood, sandalwood, citrus, tea tree, rosemary, peppermint, spearmint and frankincense. In addition, some of the essential oils can provide relaxing properties.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an antibacterial and antiviral decontamination system of the present invention in accordance with the disclosed architecture;

FIG. 2 illustrates a perspective view of one potential embodiment of an antibacterial and antiviral decontamination system of the present invention in accordance with the disclosed architecture attached to an existing HVAC and vent system of a vehicle;

FIG. 3 illustrates a perspective view of one potential embodiment of the disinfecting solution cartridge used in the antibacterial and antiviral decontamination system of the present invention in accordance with the disclosed architecture;

FIG. 4 illustrates a perspective view of one potential embodiment of the activation mechanism of the antibacterial and antiviral decontamination system of the present invention installed on a dashboard of a vehicle and being activated by a user in accordance with the disclosed architecture; and

FIG. 5 illustrates a perspective view of the interior of a bus wherein the decontaminating mist generated by the antibacterial and antiviral decontamination system of the present invention is being emitted through the existing ventilation system of the bus in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices may be shown in block diagram form in order to facilitate a description thereof. Various embodiments are discussed hereinafter. It should be noted that the figures are described only to facilitate the description of the embodiments. They are not intended as an exhaustive description of the invention, and do not limit the scope of the invention. Additionally, an illustrated embodiment need not have all the aspects or advantages shown. Thus, in other embodiments, any of the features described herein from different embodiments may be combined.

The present invention, in one exemplary embodiment, is a unique antibacterial and antiviral decontamination system integrated into the existing HVAC and ventilation system of a vehicle. The disinfecting system includes a cartridge filled with an antibacterial and antiviral solution, and a distribution system integrated directly into the HVAC and ventilation system of the vehicle that carries the antibacterial and antiviral solution. An activation switch is present on an easily accessible location, such as on dashboard, to send a trigger signal to the cartridge to release the antibacterial and antiviral solution through the distribution system, wherein the solution is dispensed as a mist through the vents to sanitize the interior of the vehicle thoroughly and completely.

As noted above, there is a long felt need in the art for an antibacterial and antiviral decontamination system that can be utilized within a vehicle, such as an airplane, train, bus, taxi, personal vehicle, etc., and that emits a disinfecting spray designed to cover the entirety of the confined space and effectively eliminate bacteria, germs, and viruses therefrom. Moreover, there is a long felt need in the art for an antibacterial and antiviral decontamination system that can be easily integrated into the existing HVAC and/or ventilation system of the vehicle, and that does not require a new infrastructure for installation or use. Additionally, there is also a long felt need in the art for an antibacterial and antiviral decontamination system that provides immediate disinfection (e.g., between flights or train departures), thereby enabling travelers to maintain proper sanitary conditions. Finally, there is a long felt need in the art for an antibacterial and antiviral decontamination system that is relatively inexpensive to manufacture and both safe and easy to use.

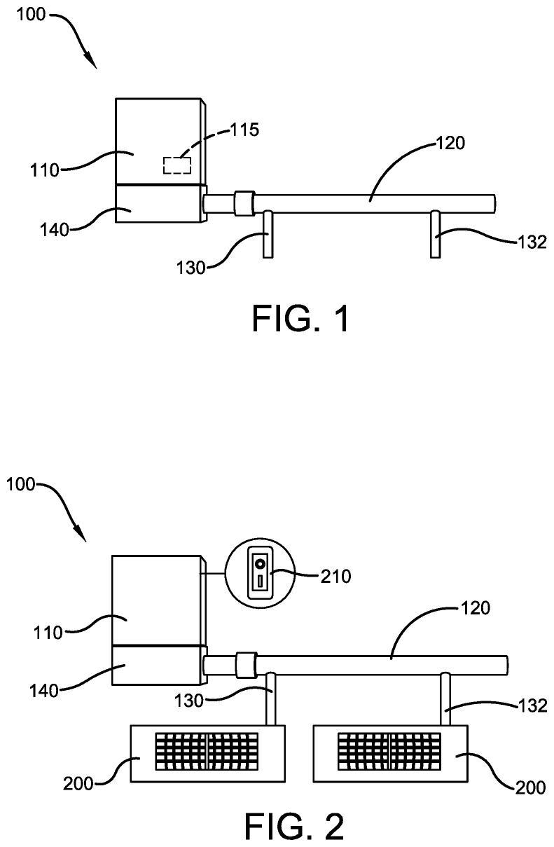

Referring initially to the drawings, FIG. 1 illustrates a perspective view of one potential embodiment of an antibacterial and antiviral decontamination system 100 of the present invention in accordance with the disclosed architecture. More specifically, the disinfecting system 100 is comprised of a cartridge 110 that contains an antibacterial and antiviral solution 115 which is used to sanitize the interior of a vehicle when dispensed. The cartridge 110 is placed in fluid communication with a base or housing 140, and is replaceable and refillable. The housing 140 may be placed at a convenient location (e.g., inside the vehicle's dashboard), or at a location where it is not visible to the user. The housing 140 is, in turn, in fluid communication with an existing HVAC or vent system of the vehicle via a central pipe or tube 120.

As best shown in FIG. 1, the central pipe 120 further branches off into, and is in fluid communication with, branch pipes 130, 132, which lead to various vents in the vehicle's cabin. As explained more fully below, once activated, the antibacterial and antiviral solution 115 is vaporized into a mist that is then transported or propelled from the housing 140 into the central pipe 120, through the various branch pipes 130, 132 and eventually through the vehicle's vents and into the passenger cabin where it effectively and quickly kills most, if not all, bacteria, viruses, germs, pathogens, microbes and the like. The ducts or pipes 120 and branches 130, 132 of the disinfecting system 100 are relatively lightweight, and may be comprised of a durable material, such as plastic, PVC or the like.

In one embodiment, the vaporizer may be present at the ends of the branches 130, 132, which is closer to the vents of the vehicle. Alternatively, a central vaporizer may be present near or in the housing 140 to create the mist of the antibacterial and antiviral solution. The various branches 120, 130, 132 of the disinfecting system 100 then carry the mist or solution to the vents of the interior of the vehicle or passenger cabin to sanitize the inside of the vehicle or passenger cabin completely.

The activation of the various components of the disinfecting system 100, such as the vaporizer or atomizer of the cartridge 110 may be powered by the internal power supply of the vehicle, or separately powered by a battery. If a battery is used, it may be a disposable battery or a rechargeable battery, and may be easily replaced or removed if necessary for recharging. Suitable batteries include alkaline, nickel-cadmium, nickel-metal hydride, etc. Throughout this specification the terms “battery”, “battery pack”, and “batteries” may be used interchangeably to refer to one or more wet or dry cells or batteries of cells in which chemical energy is converted into electricity and used as a source of DC power. References to recharging or replacing batteries may refer to recharging or replacing individual cells, individual batteries of cells, or a package of multiple battery cells as is appropriate for any given battery technology that may be used. Alternatively, the components may be powered by a separate power adaptor or through a wireless device, such as a smart phone or the like.

The disclosed disinfecting system 100 provides an easily controllable sprayer, that can produce a very fine spray or mist of antibacterial and antiviral solution. The spray molecule size is much smaller than water, thereby allowing better penetration of seats, headliners, carpets, small orifices in the ducts, blowers and other A/C system components. More specifically, the system 100 of the present invention preferably generates a solution droplet size ranging from about 5 microns to about 50 microns at a predefined dispensing rate, and an effective solution concentration of between 10 to 90%, which have proven to be most effective. More preferably, the droplet size is between 10 and 35 microns. Droplets of this size are ideal to tackle pathogens, vector carriers and pests. In addition, the effective portions of formulations can be applied in concentrations ranging from 10-90%, with the remaining portions making up propellant, stabilizers, drying agents or other non-treatment components and more preferably from 30-80% and at flow rates of up to 0.52 quarts per minute (31.7 quarts per hour or nearly 8 gallons an hour).

In one embodiment, the disinfecting system 100 can further comprise a mechanism 452 for notifying a user that the sanitization treatment is in progress, finished, or when there is a malfunction with the disinfecting system 100. The alert mechanism 452 may take several forms and can be, for example, a display with LED light(s) that show the status of the disinfecting system 100 and/or the treatment. As best shown in FIG. 4, the alert mechanism 452 may also send information about the sanitization process to an electronic device 454, such as a smartphone, that is connected to the in-vehicle Wi-Fi or through other wireless technologies such as Bluetooth, NFC, RFID, Infrared, etc.

FIG. 2 illustrates a perspective view of one potential embodiment of an antibacterial and antiviral decontamination system 100 of the present invention in accordance with the disclosed architecture and attached to an existing HVAC and vent system of a vehicle. More specifically and as described above, the disinfecting system 100 is comprised of a cartridge 110 that contains an antibacterial and antiviral solution 115 which is used to sanitize the interior of the vehicle when dispensed. The cartridge 110 is in fluid communication with each of the housing 140, the central pipe 120 and branches 130, 132.

The disinfecting system 100 is operated manually using a switch 210 at an easily accessible location, such as on the dashboard of the vehicle. When the switch 210 is flipped to ON, the disinfecting system 100 and its various components are activated by power received, for example, through the vehicle's internal power supply, and the sanitizing solution 115 is vaporized into a relatively fine mist that is used to sanitize and decontaminate the interior of the vehicle. More specifically, the sanitizing solution 115 stored within the cartridge 110 is released through the housing 140 via a nozzle 310 in the cartridge 110 and into the central pipe 120 where it is then transported to the branch pipes 130, 132 and eventually dispersed into the interior of the vehicle via the vehicle's existing ducts 200.

Once the cartridge 110 is empty, the cartridge 110 can be replaced with a new cartridge or otherwise refilled with additional sanitizing solution 115. The cartridge 110 is removably secured to the housing 140 and the central pipe 120. The cartridge 110 is also electrically connected to the controlling switch 210, and is activated when the switch 210 is flipped to the ON position. The sanitizing solution 115 stored in the cartridge 110 may also be propelled through the various ductwork 120, 130, 132 via the vehicle's existing blower, which is otherwise used to blow heated or air conditioned air through the vents 200, as is known in the art.

The antibacterial and antiviral decontamination system 100 can be used for preventative maintenance service because the system 100 can be integrated into and used with any commercial or private vehicle, regardless of year, make, or model. Further, the sanitizing solution 115 used in connection with the antibacterial and antiviral decontamination system 100 is preferably safe, non-toxic, non-corrosive and fast drying.

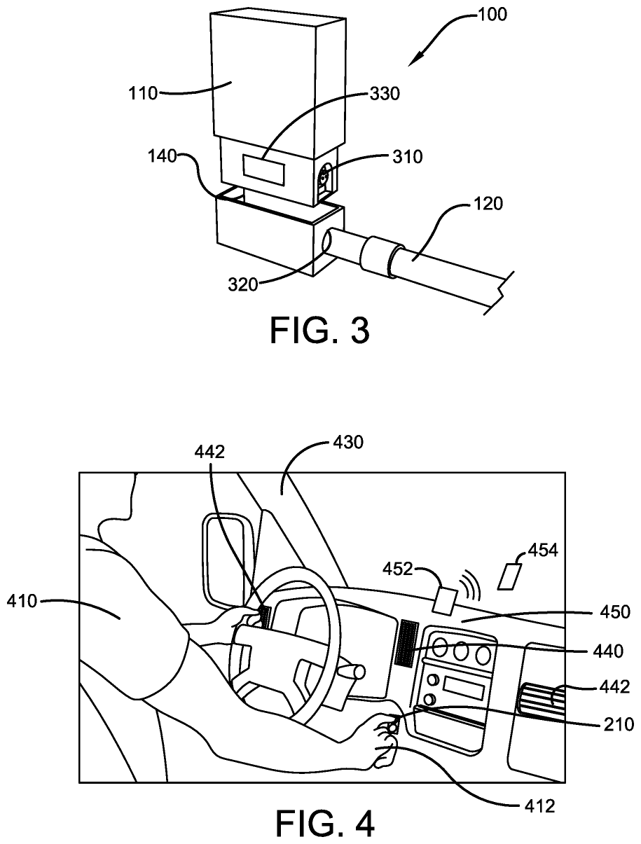

FIG. 3 illustrates a perspective view of one potential embodiment of the disinfecting solution cartridge 110 used in the antibacterial and antiviral decontamination system 100 of the present invention in accordance with the disclosed architecture. More specifically, solution cartridge 110 stores the antibacterial and antiviral decontamination solution 115 used to sanitize the interior of a vehicle, and is comprised of nozzle 310 to release the stored solution 115 through the central pipe 120 which is connected to the housing 140 at a sealed and waterproof connecting point 320. The cartridge 110 is removably secured in the housing 140, and a connection is established between the solution cartridge 110 and the central pipe or duct 120. The cartridge 110 may further comprise a built-in vaporizer 330 to vaporize the disinfecting solution 115 into a fine mist form, as described supra. The cartridge 110 also has insulating properties to maintain the properties of the solution 115 stored in the cartridge 110 in hot or cold environments, as many vehicles are stored outside and freezing may be a concern. Once vaporized, the mist generated from the disinfecting solution 115 stored in the cartridge 110 is fast acting, quick to evaporate, and is non-wetting.

FIG. 4 illustrates a perspective view of one potential embodiment of the activation mechanism or switch 210 of the antibacterial and antiviral decontamination system 100 of the present invention installed on a dashboard 450 of a vehicle 430 and being activated by a user 410 in accordance with the disclosed architecture. More specifically, the user 410 can activate the system 100 by manually flipping the controlling switch 210 located, for example, on the dashboard 450 of the vehicle 430. Alternatively, the system 100 can be activated remotely via a smartphone 454. Once activated, the vaporizer 330 vaporizes the disinfecting solution 115 stored in the cartridge 110 into a relatively fine mist that is then propelled through the nozzle 310 (e.g., by a propellant) and into the central pipe 120. Once in the central pipe 120, the mist may then be propelled through the pipe 120 and the various pipe branches 130, 132 and eventually out of the vents 440 by the vehicle's existing blower system (not shown). The solution 115 is intended to be fast-acting and quickly evaporates to reduce and possibly eliminate the transmission of viruses, bacteria, and other pathogens. It should also be appreciated that the disinfecting system 100 is built directly into the existing HVAC and ventilation system of the vehicle 430, such as an airplane cabin, train, car, bus, van, or any other vehicle.

The disinfecting system 100 may also comprise a timer (not shown) and be programmed to dispense the solution 115 into the interior of the vehicle 430 at a predetermined time, or for a pre-determined duration. The pre-determined duration may vary from about 10 to about 30 minutes, and more preferably between 15 and 20 minutes. More or less time may also be effective, depending upon the degree of cleaning (extent of contaminant load reduction) to be achieved, and the nature of the chemical cleaning agents used in the sanitizing solution 115. The sanitizing solution 115 contained in the cartridge 110 may include a formulation that contains a dissolved cleaning agent so that the air-borne mist coats and thereby cleans the surfaces, including fine surface textures, surface patterns, and tight interstitial spaces such as found, for example, in stitched seats and dash boards, etc. in the vehicle 430.

As previously stated, in one embodiment, the sanitizing solution 115 may comprise hydrogen peroxide which has good material compatibility, thereby rendering it relatively safe for use with a variety of different types of equipment and/or materials. While the present embodiment is described with particular reference to decontamination with a hydrogen peroxide vapor, it will be appreciated that other gaseous and liquid decontaminants are also contemplated, alone or in combination. The hydrogen peroxide is preferably provided in a solution ranging from about 2% to about 10% by weight of the solution. The remaining amount of the solution 115 includes water ranging from about 80% to about 95%. Fragrances, such as essential oils, may range from 2-8% of the solution 115 by weight. Essential oils may include lavender, eucalyptus, ginger, clove, cedar wood, sandalwood, citrus, tea tree, rosemary, peppermint, spearmint, frankincense, and the like. In addition, some of the essential oils may also comprise relaxing properties and/or act as insect repellants for when the vehicle operator wishes to have, for example, the windows of the vehicle open.

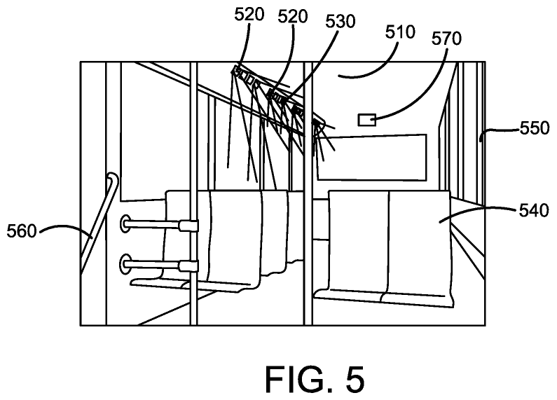

FIG. 5 illustrates a perspective view of the interior of a bus 500 wherein the decontaminating mist 530 generated by the antibacterial and antiviral decontamination system 100 of the present invention is emitted through the existing ventilation system of the bus 500 in accordance with the disclosed architecture. More specifically, when the disinfecting system 100 is activated (e.g., via the flipping of switch 210 or via the smart device 454), the sanitizing mist 530 is released from the existing vents 520 of the bus 510 and sanitizes the entire passenger area including the seats 540, handles 560 and windows 550 to reduce and possibly eliminate the transmission of viruses, bacteria, harmful microbes, germs, etc. Further, because the sanitizing solution 115 is fast drying, the bus 510 may be returned to productive use quickly and efficiently.

In one embodiment of the present invention, a method of sanitizing a passenger cabin of a commercial vehicle, such as a bus, airplane, train or the like, is disclosed. The method includes the steps of initially excluding people from the passenger cabin, and then substantially sealing the passenger cabin so that the air inside the passenger cabin is prevented from exiting the passenger cabin. Next, switching on the disinfecting system 100 of the present invention by operating a manual switch 210 (or via smart device 454). Upon activation, the antibacterial and antiviral decontamination solution 115 stored in the cartridge 110 is vaporized by the vaporizer 330 and propelled through the existing HVAC system of the vehicle and dispersed into the passenger cabin via the existing HVAC vents. In this manner, both the interior space and each of surfaces of the passenger cabin are thoroughly disinfected and sanitized.

In another embodiment of the present invention, a sensor 570, such as a movement detection sensor, proximity sensor or a video camera, is coupled to the disinfecting system 100 and is present in the passenger or travel compartment, or in a portion of the HVAC system. If the sensor 570 detects a particular movement or a passenger, an alarm (visual, audio, or other) is triggered, the disinfecting system 100 becomes inactive, and no mist is released from the ventilation system 100. The sensor 570 helps to prevent the dispersing of the sanitizing solution 115 into the passenger compartment if people are present.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “antibacterial and antiviral decontamination system”, “mist disinfection system”, “antibacterial and antiviral decontamination mist”, “antibacterial and antiviral disinfecting system”, “antibacterial and antiviral mist decontamination system”, and “disinfecting system” are interchangeable and refer to the antibacterial and antiviral decontamination system 100 of the present invention.

Notwithstanding the forgoing, the antibacterial and antiviral decontamination system 100 of the present invention can be of any suitable size and configuration as is known in the art without affecting the overall concept of the invention, provided that it accomplishes the above stated objectives. One of ordinary skill in the art will appreciate that the size, configuration and material of the antibacterial and antiviral decontamination mist system 100 as shown in the FIGS. are for illustrative purposes only, and that many other sizes of the antibacterial and antiviral decontamination system 100 are well within the scope of the present disclosure. Although the dimensions of the antibacterial and antiviral decontamination system 100 are important design parameters for user convenience, the antibacterial and antiviral decontamination system 100 may be of any size and shape that ensures optimal performance during use and/or that suits users need and/or preference.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A passenger compartment sanitizing system comprising:
a supply of a sanitizing solution;
a distribution system in fluid communication with each of the supply and a passenger compartment;
a vaporizing device for forming a mist from the sanitizing solution; and
a blower for transferring the mist through the distribution system and into the passenger compartment.

2. The passenger compartment sanitizing system as recited in claim 1, wherein the vaporizing device is one of an ultrasonic or atomizer system.

3. The passenger compartment sanitizing system as recited in claim 1, wherein the vaporizing device creates a plurality of mist particles having a size of between 5 and 50 microns.

4. The passenger compartment sanitizing system as recited in claim 1, wherein the distribution system is comprised of a main pipe and a plurality of branch pipes, each in fluid communication with the main pipe.

5. The passenger compartment sanitizing system as recited in claim 1, wherein the sanitizing solution comprises a hydrogen peroxide.

6. The passenger compartment sanitizing system as recited in claim 1, wherein the sanitizing solution comprises a hydrogen peroxide ranging from about 2% to about 10% by weight of the sanitizing solution, a quantity of water ranging from about 80% to about 95% by weight of the sanitizing solution, and a fragrance ranging from about 2% to about 8% by weight of the sanitizing solution.

7. The passenger compartment sanitizing system as recited in claim 6, wherein the fragrance is an essential oil.

8. The passenger compartment sanitizing system as recited in claim 7, wherein the essential oil is selected from a group consisting of a lavender, an eucalyptus, a ginger, a clove, a cedar wood, a sandalwood, a citrus, a tea tree, a rosemary, a peppermint, a spearmint, and a frankincense.

9. The passenger compartment sanitizing system as recited in claim 1, wherein the passenger compartment is found in an airplane, a train, a bus, a van or a motor vehicle.

10. The passenger compartment sanitizing system as recited in claim 1 further comprising an activation mechanism, wherein the activation mechanism is a select one of a switch or a smart device that is located remotely from the passenger compartment sanitizing system.

11. The passenger compartment sanitizing system as recited in claim 1, wherein the passenger compartment sanitizing generates a sanitizing solution flow rate of up to 0.52 quarts per minute.

12. A system for sanitizing travel compartments comprising:
a vehicle having a passenger compartment;
a distribution system connected to an existing HVAC system of the vehicle, wherein the HVAC system comprises a ventilation system;
a switch for activating the distribution system; and
a cartridge containing a supply of a sanitizing solution, wherein the sanitizing solution is comprised of a hydrogen peroxide.

13. A system for sanitizing travel compartments as recited in claim 12, wherein the sanitizing solution is further comprised of a quantity of water and an essential oil.

14. A system for sanitizing travel compartments as recited in claim 13, wherein the essential oil is selected from a group consisting of a lavender, an eucalyptus, a ginger, a clove, a cedar wood, a sandalwood, a citrus, a tea tree, a rosemary, a peppermint, a spearmint, and a frankincense.

15. A system for sanitizing travel compartments as recited in claim 12, wherein the distribution system is comprised of a vaporizer for vaporizing the sanitizing solution into a mist, and further wherein the vaporizer generates a mist droplet size of between 5 and 50 microns.

16. A system for sanitizing travel compartments as recited in claim 12, wherein the distribution system generates a sanitizing solution flow rate of up to 0.52 quarts per minute.

17. A system for sanitizing travel compartments as recited in claim 12, wherein the sanitizing solution is comprised of the hydrogen peroxide in a range of about 2% to about 10% of the sanitizing solution by weight, a quantity of water ranging from about 80% to about 95% of the sanitizing solution by weight, and a fragrance ranging from 2-8% of the sanitizing solution by weight.

18. An antibacterial and antiviral decontamination system comprising:
a supply of an antibacterial and antiviral solution comprised of 2-10% of a hydrogen peroxide by weight, 80-95% of a water by weight, and 2-8% of a fragrance by weight;
a distribution system in fluid communication with an HVAC system in a vehicle;
a switch for activating the antibacterial and antiviral decontamination system; and
a vaporizer in fluid communication with the distribution system, wherein the vaporizer creates a mist from the supply of an antibacterial and antiviral solution.

19. The antibacterial and antiviral decontamination system as recited in claim 18, wherein the vaporizer creates a droplet size ranging from between 5 and 50 microns.

20. The antibacterial and antiviral decontamination system as recited in claim 18, wherein the distribution system is comprised or a central line and a plurality of branch lines in fluid communication with the central line.
